(19) United States
(12) Patent Application Publication
(10) Pub. No.: US 2017/0182103 A1
(43) Pub. Date: Jun. 29, 2017

(54) Synergistic Nutritional Supplement for Absolute Energy

(71) Applicants: Tu Song-Anh Nguyen, Westminster, CA (US); Hanh Tran, Westminster, CA (US); Charles Nguyen, Westminster, CA (US); Danny Tran, Westminster, CA (US); Can Tang, Westminster, CA (US)

(72) Inventors: Tu Song-Anh Nguyen, Westminster, CA (US); Hanh Tran, Westminster, CA (US); Charles Nguyen, Westminster, CA (US); Danny Tran, Westminster, CA (US); Can Tang, Westminster, CA (US)

(21) Appl. No.: 14/981,788

(22) Filed: Dec. 28, 2015

(51) Int. Cl. A61K 36/068; A61K 31/522; A61K 33/32; A61K 31/185; A61K 36/258; A23K 20/20; A61K 31/7004; A23K 20/142; A23K 20/163; A23K 20/105; A23K 20/137; A23K 20/10; A61K 31/198; A61K 36/16

(52) U.S. Cl. CPC A61K 36/068; A61K 31/198; A61K 31/522; A61K 33/32; A61K 31/185; A61K 36/258; A61K 36/16; A61K 31/7004; A23L 1/3051; A23L 1/304; A23L 1/3002; A23L 1/30; A23K 20/142; A23K 20/163; A23K 20/105; A23K 20/137; A23K 20/10; A23K 20/30; A23V 2002/00

(57) ABSTRACT

A nutritional supplement includes a predetermined amount of an ATP increasing herbal component which includes species of cordyceps to increase a long-term internal ATP energy; a predetermined amount of L-theanine to increase a mental energy; and a predetermined amount of external energy stimulants including caffeine to provide a short-live external energy, which may be utilized as beverages, foods, supplements, and drugs so as to provide the absolute energy for all mammals including human beings, and pets such as dogs, cats, and rodents.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

Field of Invention

The present invention relates to a synergistic nutritional composition, and more particularly to an absolute energy composition which comprises Cordyceps, L-theanine, and external energy stimulants, wherein it is capable to improve a person's or a pet's well beings with an absolute energy—the internal energy of ATP (Adenosine Triphosphate increased production) and metal (mediation) energy.

Description of Related Arts

For thousands of years, people have used many kinds of external stimuli for providing extra energy for human beings. One of the most common used external stimuli is caffeine, which can be found in coffee, tea, soft drink (Coke, Mountain Dew), energy-drinks (Red Bull), caffeine supplements, and even chocolate. According to a large number of research reports, over 120,000 tons of caffeine is consumed per year worldwide, wherein caffeine counteracts the effects of adenosine, a neurotransmitter in our brain that reduces neural activity levels. Since the nerves in our brain are constantly active, we need the chemicals like adenosine to calm our brain down. So, adenosine is an important chemical to keep our brain's activity level down by inhibiting the normal neural activities, wherein it dilates our brain blood vessels so as to induce sleeping, which recharges and refreshes our brain again. It is worth mentioning that caffeine's structural configuration is similar to that of adenosine. Therefore, caffeine can bind to the same receptors of adenosine so as to prevent and decrease adenosine inhibitory actions. As a result, caffeine can counteract the effect of adenosine and makes us awake and attentive.

Therefore, caffeine gives us the extra energy, which is known as a type of external stimuli. It has many positive effects such as increasing attention and alertness and decreasing fatigue. Many studies also show that caffeine lowers the risks of having cardiovascular disease and diabetes. And, it also increases metabolic rate so as to be commonly used as thermogenic agent for weight loss products. Unfortunately, caffeine also carries many negative side effects such as anxiety, addiction, and hypertension in some people.

It is worth mentioning that people who habitually take a large amount of caffeine may have caffeine resistance, such that they need to take much more dosage than usual in order to achieve the same effect as usual. In such manner, taking overdose of caffeine may cause insomnia, nervousness and restlessness, stomach irritation, nausea and vomiting, increased heart rate and respiration, and other side effects. Seriously, taking large doses of caffeine may be unsafe and can cause irregular heartbeats and even death.

Accordingly, caffeine acts just like an external stimulus that gives us the extra energy for a short period of time. When the caffeine level wears off and releases the adenosine receptors, adenosine can work again to bind with such receptors, such that we start to feel sleepy and fatigue. Thus, caffeine is not the absolute energy that provides us with the true and constant energy that our body needs. In other words, caffeine provides us a short-term external energy. Due to the fact that the stimulating effect of the caffeine will gradually disappear with time, and the negative side effects thereof, the most effective external stimuli that can eliminate the drawbacks as explained above has to be provided and provide us with the absolute energy that every single cell of our body demands.

### SUMMARY OF THE PRESENT INVENTION

A main object of the present invention is to provide a synergistic nutritional supplement for improving a person's or pet's well beings with an absolute energy—the internal energy of ATP and metal energy, which comprises Cordyceps, L-theanine, and the external energy stimulants.

Another object of the present invention is to provide a synergistic nutritional supplement for improving a person's or pet's being with an absolute energy—the internal energy of ATP and metal energy, wherein the Cordyceps can increase the internal ATP energy within the human's body, and also increase superoxide dismutase (SOD), so as to increase endurance, energy, and alleviate many illnesses.

Another object of the present invention is to provide a synergistic nutritional supplement, wherein the L-theanine is able to provide mental energy, which can relax and clams the user's mind so as to heighten the intense concentration and improve in many cognitive function, such as memory and learning.

Another object of the present invention is to provide a synergistic nutritional supplement, which is capable of providing a trio effect of absolute energy, which are that providing an immediate and short-live external energy from caffeine, a long-term and essential energy of ATP, and a mental energy like that of meditation.

Another object of the present invention is to provide a synergistic nutritional supplement which can be used in therapeutic drugs, medication, herbal supplements, beverages, and foods for mammal including human being, dogs, cats, and rodents.

Another object of the present invention is to provide a synergistic nutritional supplement, wherein the three components, such as Cordyceps, L-theanine, and caffeine, are easy to obtain, cheap in prices, and simply to incorporating with drugs, foods, and beverages so as to provide a better nutritional supplement for improve the absolute energy.

Additional advantages and features of the invention will become apparent from the description which follows, and may be realized by means of the instrumentalities and combinations particular point out in the appended claims.

Accordingly, in order to achieve the above mentioned objects, the present invention provides a nutritional supplement, comprising:

a predetermined amount of an ATP increasing herbal component which includes species of Cordyceps having a daily dosage range from 200 mg to 2000 mg to increase a long-term internal ATP energy;

a predetermined amount of L-theanine having a daily dosage range from 10 mg to 300 mg to increase a mental energy; and

a predetermined amount of external energy stimulants, which is caffeine, so as to provide a short-live external energy.

Still further objects and advantages will become apparent from a consideration of the ensuing description and drawings.

These and other objectives, features, and advantages of the present invention will become apparent from the following detailed description, the accompanying drawings, and the appended claims.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

The true and constant energy which is required by every single cell of our body is in the form of ATP (Adenosine Triphosphate). It is well known that ATP consists of adenosine (the chemical mentioned above that makes us sleepy) plus three phosphate (A-P-P-P). And, the chemical bond between the second phosphate and the third phosphate carries a very high amount of energy, so when this bond is broken, vast amount of energy is released. In other words, ATP (A-P-P-P) gives off energy by breaking the phosphate bond to become ADP (Adenosine Diphosphate: A-P-P+P). The following shows the reaction equation:

ATP→ADP+P+Energy

Nutrients from foods are broken down into simple molecules such as fatty acids or glucose, which are absorbed in our intestine. They are further broken down and converted into the energy in our mitochondria that convert the ADP back into ATP. As shown in the below reaction equation.

ADP+P+Energy from Foods→ATP

ATP is like “cash” in our body. It stores and transfers the energy from foods to perform thousands of activities ranging from respiration to locomotion. Our muscles would be fatigue after using up the available cash ATPs. The more cash ATPs to be pumped in the cells, the higher the endurance our body can function.

All living things need energy in the form of ATP. We need ATP for energy. Our pet dog uses ATP move around. Even the fleas on our dog use the same ATP energy to jump. The problem is that the total numbers of ATPs available in our body decreases with age. By the age of 60, ATP production could be deteriorated by nearly half. When we have less ATP, we need to slow down, such that we constantly feel fatigue and tired with low vitality. Worse, our cells cannot repair themselves effectively because there is not enough ATP energy, so eventually, we have larger probability to have cancers and diseases, and finally we will die. It's just like there are so many damages in our house but not enough money to fix them. Since ATP is the internal energy inside our body, having more ATPs will give us more energy and vitality. Therefore, ATP is able to not only to keep us alive but also to give us a good quality of life free of diseases and illness.

A synergistic nutritional supplement for improving an absolute energy of all mammals according to a preferred embodiment of the present invention is illustrated, wherein the nutritional supplement comprises three components which are ATP increasing herbal component, L-theanine, and external energy stimulants.

The ATP increasing herbal component is Cordyceps, which is well known to increase the total number of ATPs in our body. Two types of Cordyceps, Cordyceps sincensis and Cordyceps militaris, are employed in the present invention, wherein daily dosage ranges of Cordyceps sincensis and Cordyceps militaris are both from 200 mg to about 2000 mg. Preferably, the daily dosage ranges of Cordyceps sincensis and Cordyceps militaris are both 1200 mg.

It is worth mentioning that Cordyceps is especially cultured in the lab possesses having high quantity of nucleosides adenine and adenosine, which are the precursors of adenosine triphosphate (ATP). Ingesting a high dose of adenosine in most animal protein does not increase the total amount of ATPs in our body. Similarly, putting the Japanese Yen in your pocket won't make you any richer unless you can convert it into US dollars. For unknown mechanism, mice, treated with Cordyceps, can convert adenosine into useful ATPs. In live study using 31P nuclear magnetic resonance (NMR) spectroscopy, mice fed with Cordyceps were shown to increase the ratio of ATP to inorganic phosphate by 47.7% after 7 days. In order words, this study showed that new ATPs were formed, and the total number of ATPs increased by almost 50% more in a merely short 7 days of treatment. Furthermore, the newly forming ATPs work the same as existing ones. Therefore, Cordyceps was not only shown to increase ATP in a short time but also ft) proven to improve exercise tolerance in long term by significantly up-regulating the skeletal muscle metabolic regulators, angiogenesis, and providing better glucose and lactate for both exercised and non-exercised rats that uptake of Cordyceps.

Accordingly, Cordyceps increases the absolute total amount of ATPs in our body; therefore, it helps many enzymes and activities from respiration to fighting infection. For example, when we have more cash, we can do more. Likewise, when we have more energy ATP, every system in our body should perform better. Cordyceps helps to increase the available energy even in the most stressful situation. For example, the Ghost moth can thrive even in an inhospitable condition such as high up in the Tibet Mountain, where none of the other insects can survive because of that they don't have the same mutually beneficial symbiosis relationship between fungus and the moth. The ghost moth is infected by some of the spores of the fungus Cordyceps and the spores of the fungus Cordyceps stay inside the body of the caterpillars, such that Cordyceps can help them to survive through the harsh winter. In such manner, most caterpillars transform into a short-life moth and reproduce in the summer. In addition, a few of them die and provide a rich growth nutrient for the Cordyceps spores to support them to become an adult fungus. Thus, both species are mutually benefited from each other. As a result, Cordyceps uses the insect body for nutrition and to spread itself everywhere. In other words, the Ghost moth species gets the needed energy ATP to thrive even in the harshest condition on earth.

Specifically, Cordyceps also increases superoxide dismutase (SOD), a natural antioxidant that converts O₂⁻ into H₂0₂ (then by catalase into harmless oxygen and water). In order to remain SOD inside humane mitochondria, manganese (a metal) is provided to support SOD to be functional and remain therewithin, such that a daily dosage range thereof is from 1 mg to about 20 mg. Preferably, the optimal daily dosage for the manganese is about 1 mg.

Generally, almost all living things use oxygen for respiration. Whenever oxygen is used, a toxic free radical superoxide O₂⁻ is formed, wherein the toxic free radical superoxide O₂⁻ could destroy anything it touches. Thus, our body needs to produce natural enzyme SOD to neutralize such toxic free radical superoxide O₂⁻. Unfortunately, the amounts of SOD along with other antioxidants decrease with age. That is the reasons why we start to have many illness when we grow older.

It should be noticed that most cancer cells do not have SOD compared to the normal healthy cells. So, when we increase the amount of SOD in our body, our normal cells can be stronger to fight against illness including cancer. With the increase of SOD and ATP by Cordyceps, it can increase endurance, energy, and alleviate opportunities for having many illnesses, especially cancer. Thus, Cordyceps increases the internal ATP energy.

According to the preferred embodiment of the present invention, the L-theanine is a natural chemical found in green tea, which is found to give mental energy, the type of energy similar to that of meditation, wherein a daily dosage range for L-theanine is from 10 mg to about 300 mg, and preferably, the daily dosage thereof is about 100 mg. The conventional sedatives cause our brain to be sluggish, cloudy, sleepy, inability to concentrate or focus, and impair overall cognitive functions, but the L-theanine doesn't. Due to the L-theanine providing the mental energy, meditation (mental energy) relaxes and calms our mind and continuously heightens it to a higher level of razor sharp focus, intense concentration, and maximizes the effects from many cognitive functions such as memory and learning. The change in neural function mentioned above can be quantitatively measured via (1) the presence of alpha-wave in EEG-electroencephalogram and (2) an increased level of GABA in the brain (just like diabetes is quantitatively measured by the blood sugar level.) Meditation produces a “mental” energy like that of a ninja, who appears relaxed but be intensely focus and able to sprint to action at any moment.

Generally speaking, a brain is continuously producing electrical currents by the neurons or brain cells, wherein these currents can be detected by a machine known as EEG (electroencephalogram), and different types of activities can be measured by this machine. For example, when a person is active under detecting by the EEG, the EEG shows a high frequency beta-wave pattern. When a person is relaxing and resting under detecting by the EEG, the EEG shows a slower alpha-wave pattern. In addition, Mediators significantly have more alpha wave than regular people. Some wave patterns (such as theta-wave occurring during the drowsy state of sleep and delta-wave occurring in the deep state of sleep) appear when we are relaxing and sleeping, such that our learning and memory abilities are limited due to our brain is shutting down. Differentially, alpha-wave is produced only when we are very relax but support us to completely focus our minds and attention, just like the state of meditation.

There are many benefits of meditation when alpha wave occurs, including lowering the stressful cortisol level. Other benefits of alpha-brain wave including relaxing thoughts and body, accessing creative state, stress reduction by lowering the cortisol level, super-learning ability, enhanced problem solving skills, having positive attitude, stabilizing emotion, natural antidepressant by increasing the serotonin level, and optimal athletic performance.

Accordingly, meditation has shown to increase the neurotransmitter GABA (gamma-aminobutyric acid), which is associated with enhanced emotional balance and improved cognition performance. GABA is an inhibitory chemical in our brain. It is like to reset your phone or computer to relieve all the unnecessary running programs so that your phone or computer can perform better. Therefore, relaxing activities such as resting and reading does not increase the GABA level, and only meditation does.

It is worth mentioning that L-theanine, an amino acid found in small quantity in green tea (Camellia sinensis), has shown to produce alpha-wave in EGG and increase GABA, just like meditation does according to many recent researches. Since many chemicals including GABA can't pass through blood-brain barrier, L-theanine can easily pass therethrough to increase the GABA level in our brain.

The external energy stimulants can be caffeine having a dosage range from 50 mg to about 600 mg, and preferably the daily dosage is about 300 mg. Moreover, the external energy stimulants can be taurine, ginseng having eleven species and ginkgo biloba, wherein the dosage range for the taurine is from 100 mg to 2000 mg, and preferably the daily dosage thereof is about 1000 mg, wherein the ginkgo biloba has a dosage range from 50 mg to 500 mg, and the daily dosage thereof is about 150 mg, wherein the ginseng with any of its eleven species has a dosage range from 100 mg to about 1000 mg, and the daily dosage thereof is about 200 mg.

It is appreciated that the synergistic nutritional supplement having Cordyceps and L-theanine, which is capable of improving a person's or a pet's well being with an absolute energy—the internal energy of ATP (Adenosine Triphosphate increased production) and mental (meditation) energy. And, the external energy stimulants incorporating with Cordyceps and L-theanine create a trio effect to improve absolute energy. The trio effect includes providing an immediate and short-live external energy from caffeine, a long-term and essential energy of ATP from Cordyceps, and a mental energy like that of meditation from L-theanine.

Accordingly, this invention is a synergistic nutritional supplement of Cordyceps and L-theanine, which may be utilized for therapeutic drugs, medication, herbal supplements in a form of tablet, capsule, and softgel, a beverage including coffee, tea, energy drinks, juices, etc., and foods including in gummies or any gelatin-based products, in cookies, cakes, and ice-cream. It is capable to improve a person's or a pet's well being with an absolute energy—the internal energy of ATP (Adenosine Triphosphate increased production) and mental (meditation) energy. It may be used with the external or stimuli energy effect of caffeine. It is intended to improve the absolute energy or the true energy of all mammals including human beings and pets such as dogs, cats, and rodents.

Caffeine and L-theanine were both shown to prevent Alzheimer's disease. More interestingly, the combination of L-theanine and caffeine yields even a better synergistic result of mood stabilizing and cognitive performance. But, some people are too sensitive to caffeine and try to pursue a substitute. Since L-theanine is so powerful in producing alpha brain wave and GABA, it has all the benefits of caffeine and meditation such as mental alertness, better focus and concentration, and faster learning but without the anxiety effects. It could be used as caffeine substitute in de-caffeinated coffee, especially at night time or for those who are too sensitive to caffeine.

As mentioned above, the nutritional supplement having Cordyceps and L-theanine can be used together without the external stimulants, such as caffeine, ginkgo biloba, taurine, and ginseng due to that some people has allergy for them. In such manner, the nutrition supplement can be used to be served in beverages including decaffeinated coffee, decaffeinated tea, juices, and flavor water so as to provide foods, beverages, or supplements for mammal pets including dogs, cats, and rodents.

In summary, the nutritional supplement further comprises D-Ribose having a dosage range from 5 gm to about 25 gm. In addition, the daily dosage thereof is about 10 gm. The composition of the nutritional supplement for improving absolute energy as shown in table 1.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

TABLE 1

| Component | | Daily Dosage |
|---|---|---|
| ATP Increasing Herbal | Cordyceps sinensis | 1200 mg |
| Component | Cordyceps militaris | 1200 mg |
| L-theanine | | 100 mg |
| External Energy Stimulants | Caffeine | 300 mg |
| | Taurine | 1000 mg |
| | Ginseng | 200 mg |
| | Ginkgo Biloba | 150 mg |
| Manganese | | 1 mg |
| D-Ribose | | 10 gm |

What is claimed is:

1. A nutritional supplement, comprising:
a predetermined amount of an ATP increasing herbal component which includes species of Cordyceps having a daily dosage range from 200 mg to 2000 mg to increase a long-term internal ATP energy;
a predetermined amount of L-theanine having a daily dosage range from 10 mg to 300 mg to increase a mental energy; and
a predetermined amount of external energy stimulants including caffeine to provide a short-live external energy.

2. The nutritional supplement, as recited in claim 1, wherein said ATP increasing herbal component can be a cordyceps sinensis.

3. The nutritional supplement, as recited in claim 1, wherein said ATP increasing herbal component can be cordyceps militaris.

4. The nutritional supplement, as recited in claim 1, wherein a daily dosage range of said ATP increasing herbal component is 1200 mg.

5. The nutritional supplement, as recited in claim 1, further comprising manganese to support an effect of a superoxide dismutase inside human mitochondria.

6. The nutritional supplement, as recited in claim 5, wherein said manganese has a daily dosage range from 1 mg to 20 mg.

7. The nutritional supplement, as recited in claim 6, wherein the daily dosage range of said manganese is 1 mg.

8. The nutritional supplement, as recited in claim 1, where the daily dosage range of said L-theanine is 100 mg.

9. The nutritional supplement, as recited in claim 1, wherein said external energy stimulant of caffeine has a dosage range from 50 mg to 600 mg.

10. The nutritional supplement, as recited in claim 9, wherein the dosage range thereof is 300 mg.

11. The nutritional supplement, as recited in claim 1, wherein said external energy stimulant is taurine having a dosage range from 100 mg to 2000 mg.

12. The nutritional supplement, as recited in claim 11, wherein the daily dosage range of said taurine is 1000 mg.

13. The nutritional supplement, as recited in claim 1, wherein said external energy stimulants is ginseng species having a dosage range from 100 mg to 1000 mg.

14. The nutritional supplement, as recited in claim 13, wherein the daily dosage of said ginseng is 200 mg.

15. The nutritional supplement, as recited in claim 1, wherein said external energy stimulants is ginkgo biloba having a dosage range from 50 mg to 500 mg.

16. The nutritional supplement, as recited in claim 15, wherein the daily dosage of said ginkgo biloba is 150 mg.

17. The nutritional supplement, as recited in claim 1, which can be used in therapeutic drug, medication, and herbal supplements in form of table, capsule, and soft gel.

18. The nutritional supplement, as recited in claim 1, which can be used in beverages and foods.

19. A nutritional supplement, comprising:
a predetermined amount of an ATP increasing herbal component which includes species of cordyceps having a daily dosage range from 200 mg to 2000 mg to increase a long-term internal ATP energy; and
a predetermined amount of L-theanine having a daily dosage range from 10 mg to 300 mg to increase a mental energy;

20. The nutritional supplement, as recited in claim 19, which can be used in beverages, foods, and supplements for mammal.

21. The nutritional supplement, as recited in claim 1, further comprising D-Ribose.

22. The nutritional supplement, as recited in claim 21, wherein said D-Ribose has a daily dosage range from 5 gm to 25 gm.

23. The nutritional supplement, as recited in claim 22, wherein the daily dosage of said D-Ribose is 10 gm.
